(19) United States
(12) Patent Application Publication
(10) Pub. No.: US 2024/0225423 A1
(43) Pub. Date: Jul. 11, 2024

(54) Nasal Anchor

(71) Applicant: Sentry Laboratories LLC, Montvale, NJ (US)

(72) Inventor: Nicholas Demetrius Tsaclas, Midlothian, VA (US)

(21) Appl. No.: 18/409,719

(22) Filed: Jan. 10, 2024

Related U.S. Application Data
(60) Provisional application No. 63/438,190, filed on Jan. 10, 2023.

(51) Int. Cl. A61B 1/00; A61B 1/267

(52) U.S. Cl. CPC A61B 1/00148; A61B 1/267

(57) ABSTRACT

A nasal anchor configured to maintain a tube of a laryngoscope in the nostril of a patient. The anchor is a cylindrical device formed of compressible foam. The device includes a longitudinal channel that is sized and shaped to grasp a laryngoscope tube. The channel is accessible through a slit. The device can be inserted onto the tube of a laryngoscope and compressed to a size sufficiently small to fit into a nostril. Once in the nostril, the anchor expands to maintain the tube secured within the nostril.

### RELATED APPLICATIONS

This application claims the benefit of pending U.S. Prov. Appl. 63/438,190 filed on Jan. 10, 2022, the contents of which are incorporated by reference herein.

### FIELD OF THE INVENTION

The invention relates to an anchor for maintaining a laryngoscope tube in a deployed position and a method of anchoring a laryngoscope using the same.

### BACKGROUND OF THE INVENTION

Laryngoscopes and similar devices are widely used by clinicians to observe patients' laryngeal tissue and surrounding areas. Deployment of a laryngoscope may sometimes require two operators, for example, when tissue is to be extracted for sampling.

### SUMMARY OF THE INVENTION

Embodiments of the invention provide an anchor that maintains the tube of a laryngoscope in the nasal passageway of a patient thereby allowing observation and tissue collection to be performed by a single clinician. Embodiments of the invention provide an expandable anchor that is configured to surround a laryngoscope tube. In use, the anchor is applied to laryngoscope tube and compressed. The laryngoscope is then deployed to a desired position within a patient. Once an optimal position is determined, the anchor is moved into the patient's nostril where it expands and maintains the laryngoscope tube in a stable position.

Embodiments of the invention further provide a method of visualizing a larynx area and sampling the same. For example, in embodiments of the invention, once the laryngoscope tube is anchored as set forth herein, a clinician may then insert an intra oral instrument that deploys a brush for gathering tissue. The intra oral instrument and extending brush may be visualized in real time by the laryngoscope.

### BRIEF DESCRIPTION OF THE DRAWINGS

Illustrative embodiments of the invention will hereafter be described with reference to the accompanying drawings, wherein like numerals denote like elements.

FIG. 1 is a front perspective view of a nasal anchor in accordance with exemplary embodiment of the invention.

FIG. 2 is a side view of a laryngoscope with a nasal anchor applied thereon in accordance with an exemplary embodiment of the invention.

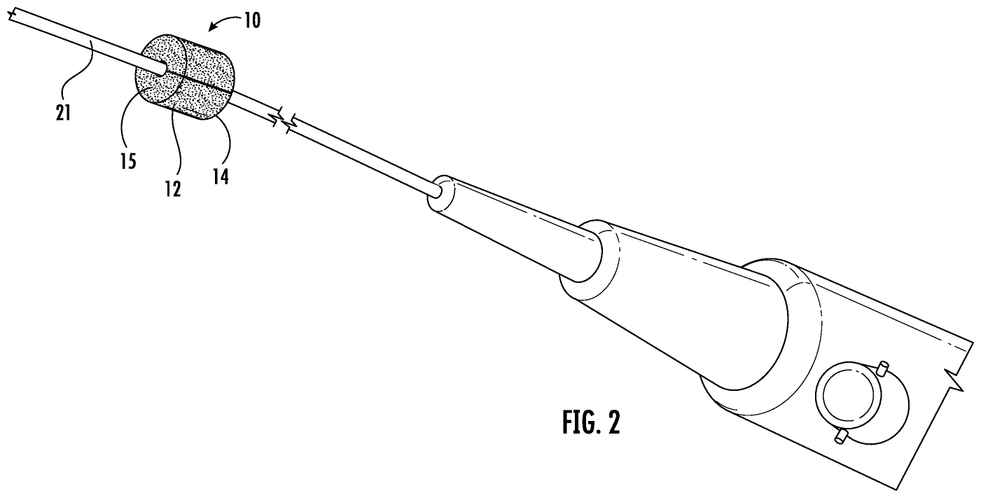

FIG. 3 is a schematic view of a laryngoscope with a nasal anchor secured thereto inserted in the nostril of a patient in accordance with an embodiment of the invention.

### DETAILED DESCRIPTION OF THE INVENTION

Embodiments of the present invention will now be described with reference to the above-identified figures of the drawings. However, the drawings and the description herein of the invention are not intended to limit the scope of the invention. It will be understood that various modifications of the present description of the invention are possible without departing from the spirit of the invention. Also, features described herein may be omitted, additional features may be included, and/or features described herein may be combined in a manner different from the specific combinations recited herein, all without departing from the spirit of the invention.

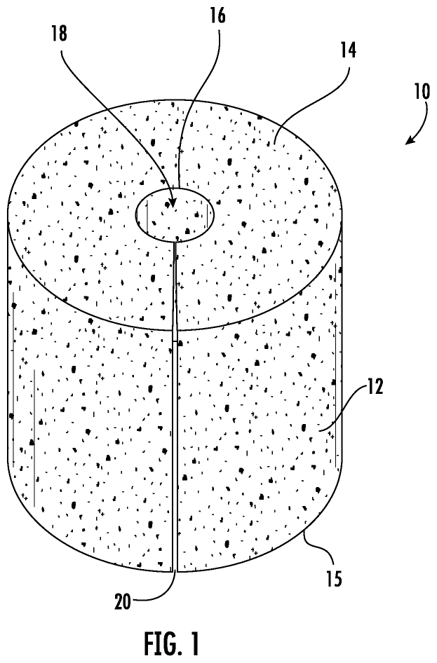

FIG. 1, shows an anchor 10 in accordance with an embodiment of the invention. As shown, anchor 10 is a generally cylindrically shaped device having an outer rounded side wall 12, and a flat circular top (and bottom) wall 14. The longitudinal axis of anchor 10 is hollow and configured for contacting an outer aspect of a laryngoscope tube. For example, as shown, a hole 16 is made in top wall 10 which creates a channel 18 through anchor 10. A mirroring hole is provided on the bottom wall 15 of anchor 10.

As further shown in FIG. 1, a slit 20 is made across a longitudinal aspect of wall 12 for allowing access to channel 18. As shown, slit 20 is an opening that extends vertically between top wall 14 and bottom wall 15—and extends laterally from rounded side wall 12 to channel 18. Opposing sections of the anchor 10 may be pulled apart at slit 20 for application on a laryngoscope tube.

Anchor 10 is filled with a compressible material such as foam or memory foam. In this regard, anchor 10 may be temporarily compressed for insertion into a nostril. The material subsequently expands to create a tight fit in the nostril canal.

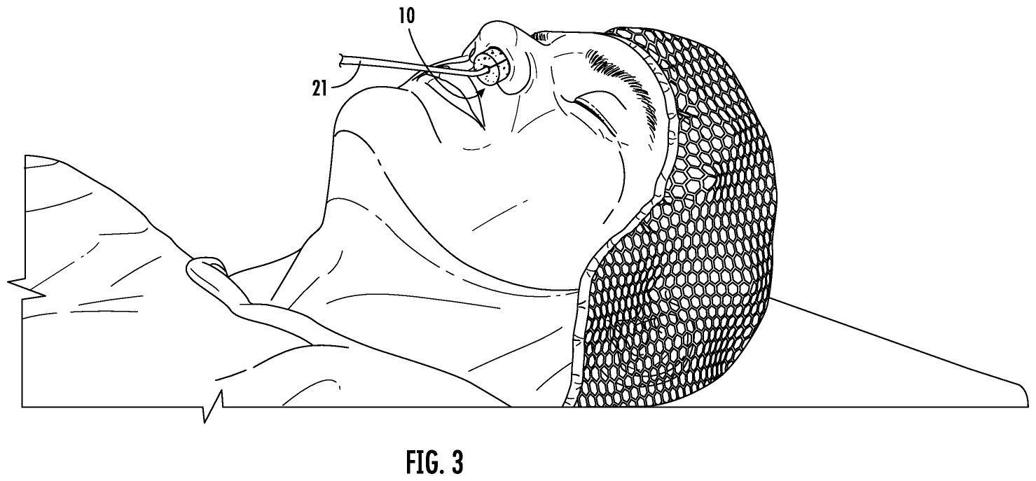

With reference to FIG. 1, in use, a physician pulls apart opposing sections of the anchor 10 a sufficient distance to apply anchor 10 to a laryngoscope tube 21. The anchor 10 is then applied such that inner walls of channel 18 contact and grip the laryngoscope tube 21. The physician then inserts the laryngoscope tube through the patient's nostril and into the laryngeal regions to be observed. With reference to FIG. 3, once a desired laryngoscope position is attained, the physician compresses the anchor and slides it up the laryngoscope tube and into the patient's nostril. Once inside the nostril, the foam is allowed to return to its uncompressed state and thereby expand to tightly grip the inner walls of the nostril.

Embodiments of the invention, thus, obviate the need for a physician to hold the laryngoscope tube in place, thereby freeing his/her hand. In addition, the anchor prevents unintended tip migration, which may occur if a patient coughs, gags, sneezes, or experiences a similar unavoidable reflexive reaction during this examination procedure.

While this invention has been described in conjunction with the embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1) A nasal anchor, comprising:
a circular top wall;
a circular bottom wall substantially parallel to the circular top wall;
a rounded wall connecting the circular top wall and the circular bottom wall;
a first hole in circular top wall;
a second hole in circular bottom wall;
a channel bridging the first hole and the second hole;
a slit in rounded wall extending from an outer surface of the rounded wall to the channel.

2) The nasal anchor of claim 1 comprising a foam filler.

3) The nasal anchor of claim 2, wherein the foam filler is memory foam.

4) The nasal anchor of claim 1, whereby the channel is sized and shaped to at least partially surround an outer tube of a laryngoscope.
